(19) United States
(12) Patent Application Publication
(10) Pub. No.: US 2022/0165367 A1
(43) Pub. Date: May 26, 2022

(54) SYSTEM AND METHOD FOR EXPLORING CHEMICAL SPACE DURING MOLECULAR DESIGN USING A MACHINE LEARNING MODEL

(71) Applicant: INTERNATIONAL INSTITUTE OF INFORMATION TECHNOLOGY, HYDERABAD, HYDERABAD (IN)

(72) Inventors: U. Deva Priyakumar, Hyderabad (IN); Sarvesh Mehta, Hyderabad (IN); Siddhartha Laghuvarapu, Hyderabad (IN); Yashaswi Pathak, Hyderabad (IN)

(21) Appl. No.: 17/526,712

(22) Filed: Nov. 15, 2021

(30) Foreign Application Priority Data
Nov. 20, 2020 (IN) 202041050608

(51) Int. Cl. G16C 20/70; G16C 20/50; G06F 30/27

(52) U.S. Cl. CPC G16C 20/70; G06F 30/27; G16C 20/50

(57) ABSTRACT

A system and method for exploring a chemical space during molecular design for at least one top hit molecule using a machine learning (ML) model are provided. The method includes (i) representing the at least one molecule stored in a drug library into at least one vector; (ii) clustering the at least one vector to obtain at least one cluster of molecules into one or more clusters; (iii) uniformly sampling a first subset of molecules from each cluster of molecules; (vi) determining a docking score for sampled subset of molecules; (iv) training the ML model by correlating sampled subset of molecules with docking score; (viii) computing acquisition function values for a second subset of molecules from each cluster; and (ix) determining at least one top hit molecule based on the computed acquisition function values, thereby exploring the chemical space for the at least one top hit molecule.

### CROSS-REFERENCE TO PRIOR-FILED PATENT APPLICATIONS

This application claims priority from the Indian provisional application no. 202041050608 filed on Nov. 20, 2020, which is herein incorporated by reference.

### TECHNICAL FIELD

The embodiments herein generally relate to exploring a chemical space during molecular design, and more particularly, to a system and method for exploring a chemical space by determining a set of top hit molecules using a machine learning model during molecular design.

### DESCRIPTION OF THE RELATED ART

In many areas like medicine, biotechnology, and pharmacology, drug discovery is a process by which new medication is discovered. In the process of drug discovery, chemical libraries are used to screen compounds that are usable in industrial processes. The chemical libraries include a series of stored chemical compounds. Each chemical compound is associated with information such as chemical structure, purity, quantity, and physiochemical characteristics of the chemical compound. Hence, these chemical libraries are extremely huge. Evaluation of each molecule in the chemical libraries is computationally infeasible.

Existing systems initially identify a drug target and validate the drug target. Followed by validation of the drug target, the existing system identifies hit molecules with a high binding affinity (drug-like molecules) against the drug target using computational techniques. The identified hit molecules are evaluated typically based on biochemical assays towards lead identification. Further, processes include lead optimization, in vitro evaluation, and in vivo evaluation. Before a drug is approved for use, pre-clinical studies and clinical trials are implemented. Hence the existing systems follow an expensive and time-consuming process.

Therefore, there arises a need to address the aforementioned technical drawbacks in existing technologies in exploring a chemical space for molecules.

### SUMMARY

In view of foregoing an embodiment herein provides a processor-implemented method for exploring a chemical space for at least one molecule during molecular design using a machine learning model. The method includes the steps of (i) selecting the at least one molecule that is stored in a drug library and representing, using a vector representation technique, the at least one molecule as at least one vector; (ii) clustering, using at least one clustering technique, the at least one vector corresponding to the at least one molecule to obtain one or more clusters of the at least one molecule; (iii) uniformly sampling a first subset of molecules from each cluster of molecules; (iv) determining, using a computational technique, a docking score for sampled subset of molecules, the docking score determines an acquisition function of the at least one molecule based on the sampled subset of molecules; (v) training, using a gaussian process, the machine learning model by correlating the sampled subset of molecules with the determined docking score of the at least one molecule to obtain a trained machine learning model; (vi) computing, using the trained machine learning model, the acquisition function values for a second subset of molecules from each cluster of the at least one molecule; and (vii) determining at least one top hit molecule for the at least one molecule based on the computed acquisition function values of the second subset of molecules to explore the chemical space for the at least one top hit molecule.

In some embodiments, representing the at least one molecule into the at least one vector by, (i) extracting a substructure for the at least one molecule at radii 0 and 1 and assigning a unique identifier to the at least one molecule; (ii) representing the at least one molecule as a sentence, using an assigned unique identifier to the at least one molecule; and (iii) encoding words in the sentence for the at least one molecule into the at least one vector using an unsupervised machine learning model, the unsupervised machine learning model is trained by correlating the words for the at least one molecule and the at least one vector.

In some embodiments, obtaining, using a computational technique, the docking score for each molecule of the sampled subset of molecules by, (i) obtaining a structure for the ligand using a dataset, the dataset is obtained from a database, the ligand is an ion or a molecule that binds to a target protein; (ii) obtaining the target protein from a protein database; (iii) performing protein-ligand docking for obtained structure for the ligand and obtained target protein to generate grid maps, electron density, and desolvation maps for each type of atom of each molecule of the sampled subset of molecules; and (iv) computing the docking score for each molecule of the sampled subset of molecules based on generated grid maps, electron density, and desolvation maps for each type of atom.

In some embodiments, computing the acquisition function for the second set of molecules based on an upper confidence bound, an expected improvement, a probability of improvement obtained from the gaussian process.

In some embodiments, sampling the first subset of molecules uniformly by selecting the at least one top hit molecule based on the value of the acquisition function for the set of molecules.

In some embodiments, retraining the machine learning model when convergence criteria are not met, the convergence criteria include a maximum number of allowable docking scores for the sampled subset of molecules.

In one aspect, one or more non-transitory computer-readable storage medium store the one or more sequence of instructions, which when executed by a processor, further causes a method for exploring a chemical space for at least one molecule during molecular design using a machine learning model. The method includes the steps of (i) selecting the at least one molecule that is stored in a drug library and representing, using a vector representation technique, the at least one molecule as at least one vector; (ii) clustering, using at least one clustering technique, the at least one vector corresponding to the at least one molecule to obtain one or more clusters of the at least one molecule; (iii) uniformly sampling a first subset of molecules from each cluster of molecules; (iv) determining, using a computational technique, a docking score for sampled subset of molecules, the docking score determines an acquisition function of the at least one molecule based on the sampled subset of molecules; (v) training, using a gaussian process, the machine learning model by correlating the sampled subset of molecules with the determined docking score of the at least one molecule to obtain a trained machine learning model; (vi) computing, using the trained machine learning model, the acquisition function values for a second subset of molecules from each cluster of the at least one molecule; and (vii) determining at least one top hit molecule for the at least one molecule based on the computed acquisition function values of the second subset of molecules to explore the chemical space for the at least one top hit molecule.

In another aspect, a system for exploring a chemical space for at least one molecule during molecular design using a machine learning model. The system includes a server that is communicatively coupled with a user device associated with a user. The server includes a memory that stores a set of instructions and a processor that executes the set of instructions and is configured to (i) select the at least one molecule that is stored in a drug library and representing, using a vector representation technique, the at least one molecule as at least one vector; (ii) cluster, using at least one clustering technique, the at least one vector corresponding to the at least one molecule to obtain one or more clusters of the at least one molecule; (iii) uniformly sample a first subset of molecules from each cluster of molecules; (iv) determine, using a computational technique, a docking score for sampled subset of molecules, the docking score determines an acquisition function of the at least one molecule based on the sampled subset of molecules; (v) train, using a gaussian process, the machine learning model by correlating the sampled subset of molecules with the determined docking score of the at least one molecule to obtain a trained machine learning model; (vi) compute, using the trained machine learning model, the acquisition function values for a second subset of molecules from each cluster of the at least one molecule; and (vii) determine at least one top hit molecule for the at least one molecule based on the computed acquisition function values of the second subset of molecules to explore the chemical space for the at least one top hit molecule.

In some embodiments, representing the at least one molecule into the at least one vector by, (i) extracting a substructure for the at least one molecule at radii 0 and 1 and assigning a unique identifier to the at least one molecule; (ii) representing the at least one molecule as a sentence, using an assigned unique identifier to the at least one molecule; and (iii) encoding words in the sentence for the at least one molecule into the at least one vector using an unsupervised machine learning model, the unsupervised machine learning model is trained by correlating the words for the at least one molecule and the at least one vector.

In some embodiments, obtaining, using a computational technique, the docking score for each molecule of the sampled subset of molecules by, (i) obtaining a structure for the ligand using a dataset, the dataset is obtained from a database, the ligand is an ion or a molecule that binds to a target protein; (ii) obtaining the target protein from a protein database; (iii) performing protein-ligand docking for obtained structure for the ligand and obtained target protein to generate grid maps, electron density, and desolvation maps for each type of atom of each molecule of the sampled subset of molecules; and (iv) computing the docking score for each molecule of the sampled subset of molecules based on generated grid maps, electron density, and desolvation maps for each type of atom.

In some embodiments, computing the acquisition function for the second set of molecules based on an upper confidence bound, an expected improvement, a probability of improvement obtained from the gaussian process.

In some embodiments, sampling the first subset of molecules uniformly by selecting the at least one top hit molecule based on the value of the acquisition function for the set of molecules.

In some embodiments, retraining the machine learning model when convergence criteria are not met, the convergence criteria include a maximum number of allowable docking scores for the sampled subset of molecules.

The system and method for maximizing the exploration of chemical space during molecular design are evaluated by considering a small portion of the molecular dataset. The present method improves by reducing computation time in finding top hits in vast chemical space. The present method is less expensive as it evaluates the top-performing molecules in the molecular dataset.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The embodiments herein will be better understood from the following detailed description with reference to the drawings, in which:

FIG. 1 is a block diagram that illustrates a system for exploring a chemical space during molecular design for at least one top hit molecule using a machine learning model, according to some embodiments herein;

FIG. 2 is a block diagram that illustrates a server of FIG. 1, according to some embodiments herein;

FIG. 3 illustrates an exemplary process of representing a set of molecules as one or more vectors using the server of FIG. 1, according to some embodiments herein;

FIG. 4 illustrates an exemplary process of constructing a unique identifier-vector lookup table for the one or more vectors using the server of FIG. 1, according to some embodiments herein;

FIG. 5 illustrates an exemplary diagram of exploring a chemical space for at least one set of top hit molecules during molecular design using a machine learning model according to some embodiments herein;

FIGS. 6A-6B illustrate graphical representations of mean docking score compared with top hit molecules for target protein TTBK, and for target protein CoV-2 M^{pro}, according to some embodiments herein;

FIGS. 7A and 7B illustrate graphical representations of a fraction of top 500 sampled molecules that are the actual top hit molecules against a percentage of samples for target protein TTBK, and target protein CoV-2 M^{pro}, using Zinc-250K drug library according to some embodiments herein;

FIG. 8 illustrates a graphical representation of the fraction of top 500 sampled molecules that are the actual top hit molecules against a percentage of samples for target protein TTBK using enamine drug library according to some embodiments herein;

FIG. 9A illustrates a graphical representation of the fraction of top 500 sampled molecules that are the actual top hit molecules against a percentage of samples for target protein AmpC using ultra-large drug library according to some embodiments herein;

FIG. 9B illustrates a distribution plot of docking scores for top 1000 hit molecules for target protein AmpC using ultra-large drug library according to some embodiments herein;

FIG. 10 is a flow diagram that illustrates a method for exploring a chemical space for at least one molecule during molecular design using a machine learning model, according to some embodiments herein; and

FIG. 11 is a schematic diagram of a computer architecture in accordance with the embodiments herein.

### DETAILED DESCRIPTION

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

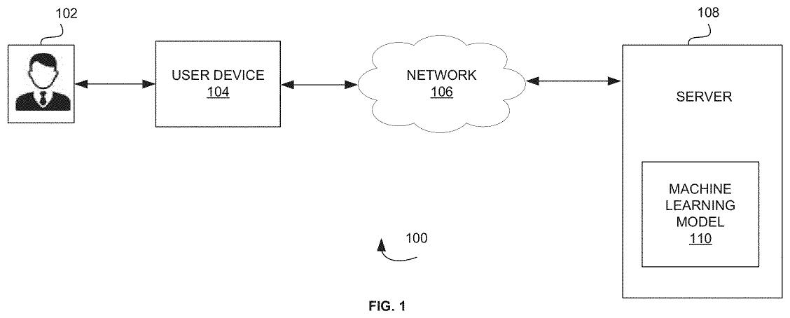

As mentioned, there is a need for a system and method for exploring a chemical space using a machine learning model. The embodiments herein are achieved by proposing a system and method for exploring a chemical space by identifying at least one set of top hit molecules using a machine learning model. Referring now to the drawings, and more particularly to FIG. 1 through FIG. 11, where similar reference characters denote corresponding features consistently throughout the figures, preferred embodiments are shown.

FIG. 1 is a block diagram that illustrates a system 100 for exploring a chemical space during molecular design for at least one set of top hit molecules using a machine learning model 110, according to some embodiments herein. The system 100 includes a user device 104, and a server 108. The user device 104 may be associated with the user 102. The user 10 device 102 includes a user interface to obtain an input from the user 102 to explore chemical space during the molecular design of a drug. The user device 104 includes, but is not limited to, a handheld device, a mobile phone, a kindle, a Personal Digital Assistant (PDA), a tablet, a laptop, a music player, a computer, an electronic notebook, or a smartphone and the like. The server 108 includes a device processor and a non-transitory computer-readable storage medium storing one or more sequences of instructions, which when executed by the device processor causes enablement to explore a chemical space for at least one top hit molecule using the machine learning model 110. The server 108 may receive the input to explore chemical space during the molecular design of a drug from the user device 104 through a network 106. The network 106 includes, but is not limited to, a wireless network, a wired network, a combination of the wired network and the wireless network or Internet, and the like. In some embodiments, the system 100 may include an application that may be installed in android based devices, windows-based devices, or any such mobile operating systems devices for exploring the chemical space during the molecular design of the drug.

The server 108 indicates all molecules in a drug library after receiving the input from the user device 104. The input may include a set of molecules and a constant number. The server 108 may select at least one molecule that is stored in a drug library. The server 108 represents the at least one molecule into at least one vector using a vector representation technique. The vector representation technique may include at least one of an extended connectivity fingerprint (ECFP), continuous and data-driven descriptors (CDDD), or a mol2vec. The server 108 may use the ECFP molecular embedding technique or the mol2vec embedding technique to encode the at least one molecule into at least one vector.

The server 108 clusters the at least one vector corresponding to the at least one molecule to obtain one or more clusters of the at least one molecule using a clustering technique. In some embodiments, the clustering technique is a K means clustering.

The server 108 may select at least one vector of the at least one molecule from each cluster based on the constant number. The server 108 samples a first subset of molecules uniformly to obtain a sampled subset of molecules. The sampled subset of molecules may be defined by the user 102.

The server 108 determines, using a computational technique, a docking score for each of the sampled subset of molecules. The docking score is a scoring function used to predict binding affinity of a ligand and a targeted molecule. Alternatively, the docking score for each of the sampled subset of molecules may also be obtained from experimental methods. In some embodiments, the computational technique may be a protein-ligand docking method. The docking score determines an acquisition function of the at least one molecules based on the sampled subset of molecules.

The protein-ligand docking method involves (i) obtaining a structure of a ligand using a dataset (ii) obtaining a target protein from a protein data bank, (iii) performing protein-ligand docking and generates grid maps for each atom type along with electron density maps and desolvation maps, and (iv) calculates the docking score of ligand and target protein. In some embodiments, the dataset may be obtained from a database.

The server 108 trains the machine learning model 110 by correlating the sampled subset of molecules with the determined docking score of the at least one molecule to obtain a trained machine learning model. In some embodiments, the server 108 may use a Gaussian process, or a deep Gaussian process to train the machine learning model 110. The server 108 computes using the trained machine learning model, an acquisition function for a second subset of molecules from each cluster of the at least one molecule. The server 108 determines the at least one top hit molecule from the set of molecules for the at least one molecule based on the computed acquisition function values of the second subset of molecules, thereby exploring the chemical space for the at least one top hit molecule.

In some embodiments, the machine learning model is retrained when convergence criteria are not met, the convergence criteria include a maximum number of allowable docking scores for the sampled subset of molecules.

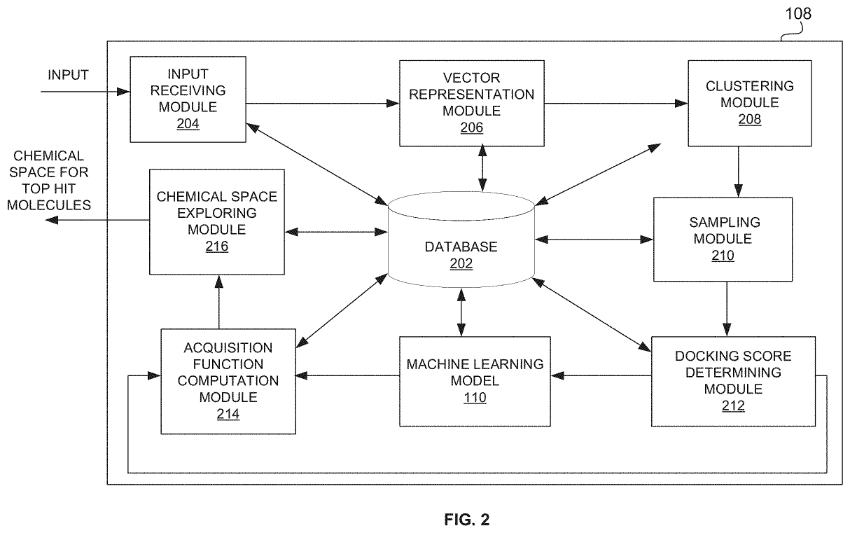

FIG. 2 is a block diagram that illustrates a server 108 of FIG. 1, according to some embodiments herein. The server 108 includes a database 202, an input receiving module 204, a vector representation module 206, a clustering module 208, a sampling module 210, a docking score determining module 212, a machine learning model 110, an acquisition function computing module 214, and a chemical space exploring module 216. The input receiving module 204 receives an input to explore a chemical space during the molecular design of a drug. The input may be received from the user 102 through the user device 104. The input may include a set of molecules and a constant number. The database 202 stores the input obtained from the user 102. The server 108 may select at least one molecule from a drug library based on the input received. The vector representation module 206 represents the at least one molecule stored in a drug library into at least one vector using a vector representation technique. The vector representation module 206 represents the at least one molecule into at least one vector by, (i) extracting a substructure for at least one molecule at radii 0 and 1 and assigning a unique identifier to each molecule; (ii) representing the at least one molecule as a sentence, using an assigned unique identifier to each molecule; and (iii) encoding words in the sentence for the at least one molecule into the at least one vector using an unsupervised machine learning model.

The clustering module 208 clusters, using a clustering technique, the at least one vector of the at least one molecule into one or more clusters. In some embodiments, the clustering technique may be a K means clustering. The clustering module 208 automatically selects a number of the vectors of the at least one molecule from each cluster to obtain a subset of molecules based on the constant number of the input. The sampling module 210 samples a first subset of molecules uniformly to obtain sampled subset of molecules. The subset of molecules for sampling may be defined by user 102.

The docking score determining module 212 determines, using a computational technique, a docking score for each of the sampled subset of molecules. Alternatively, the docking score for each of the sampled subset of molecules may also be obtained from experimental methods. In some embodiments, the computational technique may be a protein-ligand docking method. For docking, the docking score determining module 212 prepares a selected ligand. The selected ligand may be a target protein TTBK1, a target protein AmpC, a target protein CoV-2 M^{pro}.

The machine learning model 110 is trained by correlating sampled subset of molecules with the determined docking score of the at least one molecule to obtain a trained machine learning model. In some embodiments, the server 108 may use a Gaussian process, or a deep Gaussian process to train the machine learning model 110.

In some embodiments, the machine learning model is retrained when convergence criteria are not met, the convergence criteria include a maximum number of allowable docking scores.

The acquisition function computing module 214 computes, using the machine learning model 110, an acquisition function for a set of molecules that are present in the drug library for the input received. In some embodiments, computing the acquisition function based on values of a gaussian process upper confidence bound, an expected improvement, a probability of improvement. In some embodiments, sampling a subset of molecules uniformly by selecting the top hit molecules based on the value of the acquisition function.

The acquisition function computing module 214 computes an acquisition function for a second set of molecules that are present in the drug library. In some embodiments, the acquisition function may be based on an expected improvement technique upper confidence bound or probability of improvement. The acquisition function computing module 214 obtains acquisition function values for the new molecules. In some embodiments, the acquisition function computing module 214 returns an appended dataset with new molecules if convergence criteria are not met.

The chemical space exploring module 216 explores the chemical space for at least one top hit molecule based on a value of the acquisition function for the set of molecules using the trained machine learning model.

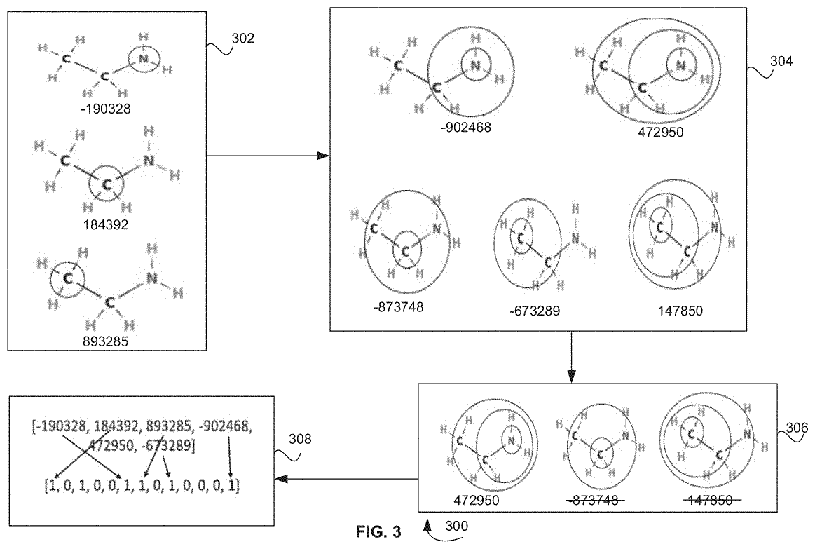

FIG. 3 illustrates an exemplary process of representing a set of molecules as one or more vectors using the server 108 of FIG. 1, according to some embodiments herein. In the exemplary process 300, at a step 302, an integer value for each atom in the set of molecules is assigned. For example, at the step 302, one of the molecules is assigned with the integer value of −190328 as shown in FIG. 3. The representation of the one or more vectors of the set of molecules optimizes the exploration of chemical space for the set of molecules. The server 108 assigns an integer value for each atom in the set of molecules. Based on the integer value, and bond information of an atom identifier, the server 108 augments the exploration of chemical space to obtain a unique identifier for each atom in the set of molecules. The server 108 iterates augmentation of the exploration of the chemical space to indicate the depth of the bond information at each atom center. The server 108 removes duplicates of substructures in the case of the substructures with multiple unique identifiers. The server 108 constructs the substructures into a bit vector for each atom in the set of molecules. In the exemplary process 300 at a step of 304, the integer value for each atom based on bond information to obtain a unique identifier is augmented. For example, at the step 304, one of the molecules augments the integer value from −190328 to −902468 as shown in FIG. 3. In the exemplary process 300 at a step of 306, duplicates of multiple unique identifiers in the case of substructures at 306 are removed. For example, at the step 306 duplicates of a substructure with unique identifier −873748 are removed as shown in FIG. 3. In the exemplary process 300 at a step of 304, the substructures into a bit vector are constructed. For example, the substructure with unique identifier −190328 has a bit vector of 0, 1.

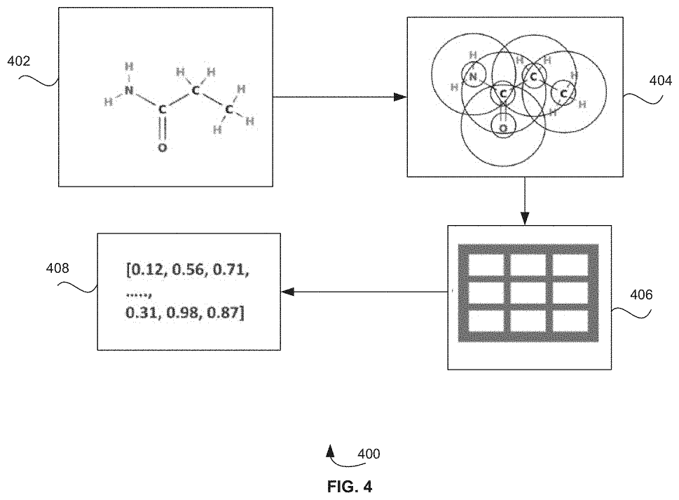

FIG. 4 illustrates an exemplary process of constructing a unique identifier-vector lookup table for the one or more vectors using the server 108 of FIG. 1, according to some embodiments herein. The server 108 arranges sequences of molecules using unique identifiers of each atom in a set of molecules. The server 108 constructs a unique identifier-vector lookup table. For a new molecule, an embedding is obtained by summing vectors of all the unique identifiers in a unique identifier-vector lookup table. In the exemplary process at a step 402, a new molecule is obtained. In the exemplary process at a step 404, an extracted substructure for the new molecule is included. In the exemplary process at a step 406, a unique identifier-vector lookup table is included. In the exemplary process at a step 408, embeddings of all the unique identifiers in the unique identifier-vector lookup table are included.

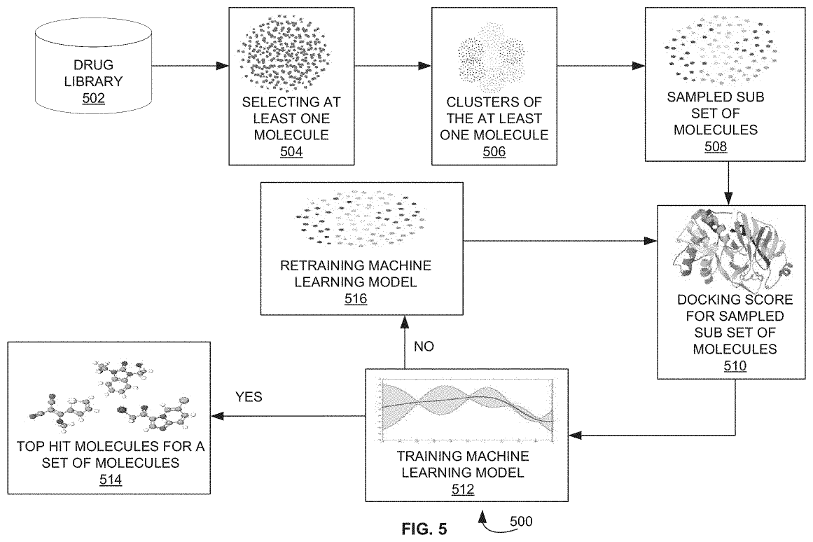

FIG. 5 illustrates an exemplary diagram 500 of exploring a chemical space for at least one set of top hit molecules during molecular design using a machine learning model 110 according to some embodiments herein. The exemplary diagram 500 includes selecting the at least one molecule from a drug library 502 at a step 504. The exemplary diagram 500 includes representing the at least one molecule into at least one vector and clustering, using at least one clustering technique, the at least one vector to obtain at least one cluster of molecules into one or more clusters at step 506. The exemplary diagram 500 includes uniformly sampling a first subset of molecules from each cluster of molecules at step 508. The exemplary diagram 500 includes determining a docking score for a sampled subset of molecules at step 510. The exemplary diagram 500 includes training a machine learning model by correlating the sampled subset of molecules with the determined docking score of the at least one molecule at step 512. The exemplary diagram 500 includes determining, using the trained machine learning model, the at least one set of top hit molecules from the set of molecules for the at least one molecule at step 514. The exemplary diagram 500 includes retraining the machine learning model when a maximum number of allowable docking scores for the sampled subset of molecules are not met.

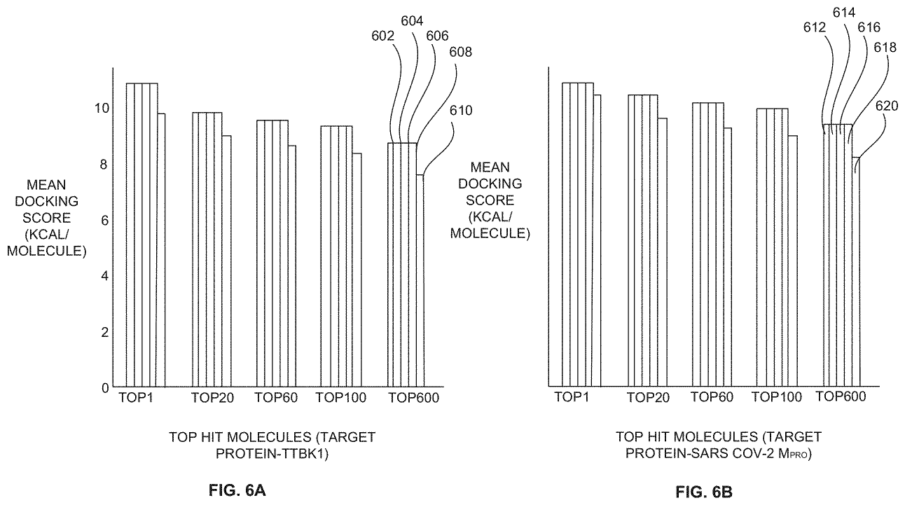

FIGS. 6A and 6B illustrate graphical representations of mean docking score compared with top hit molecules for target protein TTBK, and target protein CoV-2 M^{pro}, according to some embodiments herein. FIG. 6A illustrates a graphical representation of the mean docking score compared with top hit molecules for target protein TTBK. The graphical representation depicts the mean docking score for target protein TTBK on the Y-axis and top hit molecules for target protein TTBK1 on the X-axis. At 602, the graph represents the mean docking score for the top 600 hit molecules in the drug library, when the top 600 hit molecules are sampled using the whole dataset for target protein TTBK1. At 604, the graph represents the mean docking score for the top 600 hit molecules in the drug library, when the top 600 hit molecules are sampled using Mol2Vec for target protein TTBK1. At 606, the graph represents the mean docking score for the top 600 hit molecules in the drug library, when the top 600 hit molecules are sampled using continuous and data-driven descriptors (CDDD) for target protein TTBK1. At 608, the graph represents the mean docking score for the top 600 hit molecules in the drug library, when the top 600 hit molecules are sampled using extended connectivity fingerprint (ECFP) for target protein TTBK1. At 610, the graph represents the mean docking score for the top 600 hit molecules in the drug library, when the top 600 hit molecules are sampled using a random sampling method for target protein TTBK1.

FIG. 6B illustrates a graphical representation of the mean docking score compared with top hit molecules for target protein CoV-2 M^{pro}. The graphical representation depicts the mean docking score for target protein TTBK on the Y-axis and top hit molecules for target protein CoV-2 M^{pro} on the X-axis. At 612, the graph represents the mean docking score for the top 600 hit molecules in the drug library, when the top 600 hit molecules are sampled using the whole dataset for target protein CoV-2 M^{pro}. At 614, the graph represents the mean docking score for the top 600 hit molecules in the drug library, when the top 600 hit molecules are sampled using Mol2Vec for target protein CoV-2 M^{pro}. At 616, the graph represents the mean docking score for the top 600 hit molecules in the drug library, when the top 600 hit molecules are sampled using continuous and data-driven descriptors (CDDD) for target protein CoV-2 M^{pro}. At 618, the graph represents the mean docking score for the top 600 hit molecules in the drug library, when the top 600 hit molecules are sampled using extended connectivity fingerprint (ECFP) for target protein CoV-2 M^{pro}. At 620, the graph represents the mean docking score for the top 600 hit molecules in the drug library, when the top 600 hit molecules are sampled using a random sampling method for target protein CoV-2 M^{pro}.

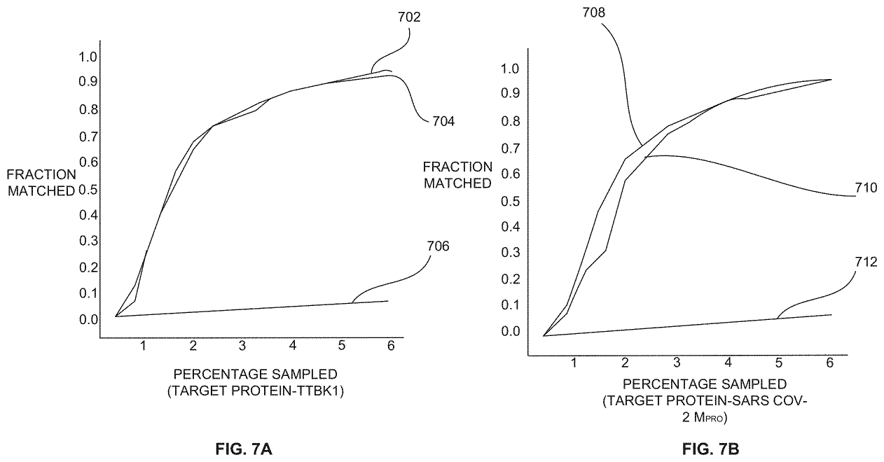

FIGS. 7A and 7B illustrate graphical representations of a fraction of top 500 sampled molecules that are the actual top hit molecules against a percentage of samples for target protein TTBK, and target protein CoV-2 M^{pro}, using Zinc-250K drug library according to some embodiments herein. FIG. 7A illustrates a graphical representation of a fraction of the top 500 sampled molecules that are the actual top hit molecules against a percentage of samples for target protein TTBK. The graphical representation depicts the fraction of the top 500 sampled molecules that are the actual top hit molecules on the Y-axis and the percentage of samples for target protein TTBK, on X-axis. At 702, the graph represents a fraction of the top 500 sampled molecules that are the actual top hit molecules using a trained machine learning model for target protein TTBK. At 704, the graph represents a fraction of the top 500 sampled molecules that are the actual top hit molecules using a machine learning model for target protein TTBK. At 706, the graph represents a fraction of the top 500 sampled molecules that are actual top hit molecules using a random model for target protein TTBK.

FIG. 7B illustrates a graphical representation of the fraction of top 500 sampled molecules that are the actual top hit molecules against a percentage of samples for target protein CoV-2 M^{pro}. The graphical representation depicts the fraction of the top 500 sampled molecules that are the actual top hit molecules on the Y-axis and the percentage of samples for target protein CoV-2 M^{pro}, on X-axis. At 708, the graph represents a fraction of the top 500 sampled molecules that are the actual top hit molecules using a trained machine learning model for target protein CoV-2 M^{pro}. At 710, the graph represents a fraction of the top 500 sampled molecules that are the actual top hit molecules using a machine learning model for target protein CoV-2 M^{pro}. At 712, the graph represents the fraction of the top 500 sampled molecules that are the actual top hit molecules using a random model for target protein CoV-2 M^{pro}.

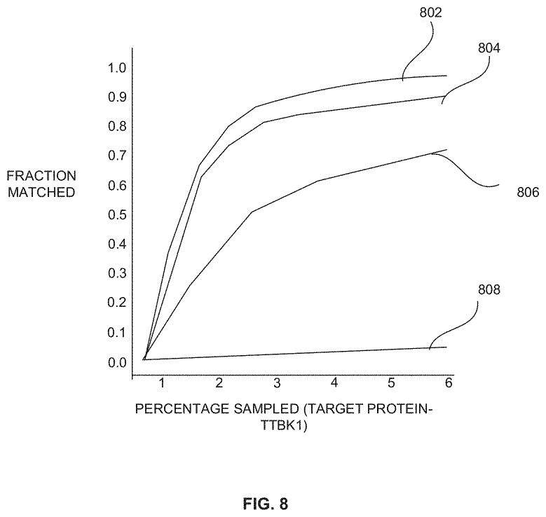

FIG. 8 illustrates a graphical representation of the fraction of top 500 sampled molecules that are the actual top hit molecules against a percentage of samples for target protein TTBK using enamine drug library according to some embodiments herein. The graphical representation depicts the fraction of the top 500 sampled molecules that are the actual top hit molecules using enamine drug library on Y-axis and percentage of samples for target protein TTBK1, on X-axis. The enamine drug library includes 2,106,952 screening compounds. The trained machine learning model is applied to enamine the drug library to explore chemical space by identifying one set of top hit molecules by docking against the target protein TTBK1. At 802, the graph represents the fraction of the top 500 sampled molecules that are the actual top hit molecules when the top 500 hit molecules are sampled using the whole dataset for target protein TTBK1. At 804, the graph represents the fraction of the top 500 sampled molecules that are the actual top hit molecules, when the top 500 hit molecules are sampled using Mol2Vec for target protein TTBK1. At 806, the graph represents the fraction of the top 500 sampled molecules that are the actual top hit molecules when the top 500 hit molecules are sampled using continuous and data-driven descriptors (CDDD) for target protein TTBK1. At 808, the graph represents the fraction of the top 500 sampled molecules that are the actual top hit molecules when the top 500 hit molecules are sampled using extended connectivity fingerprint (ECFP) for target protein TTBK1. At 810, the graph represents the fraction of the top 500 sampled molecules that are the actual top hit molecules when the top 500 hit molecules are sampled using a random sampling method for target protein TTBK1.

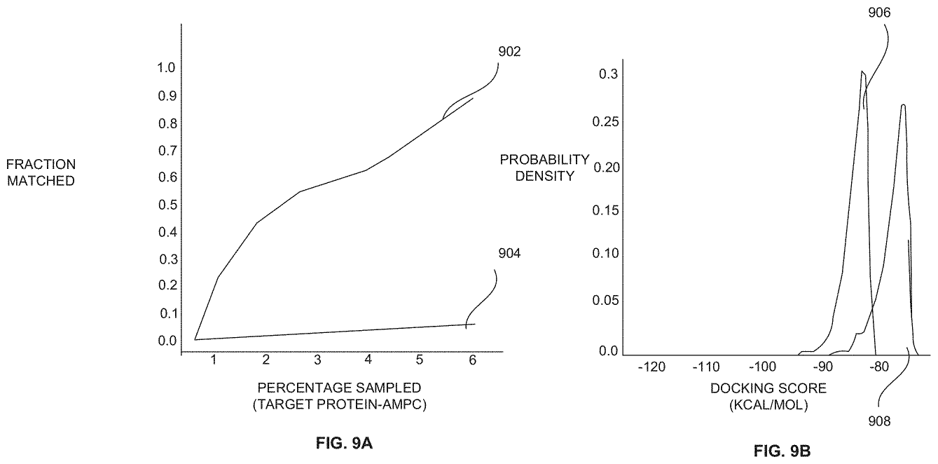

FIG. 9A illustrates a graphical representation of the fraction of top 500 sampled molecules that are the actual top hit molecules against a percentage of samples for target protein AmpC using an ultra-large drug library according to some embodiments herein. The graphical representation depicts the fraction of the top 500 sampled molecules that are the actual top hit molecules using ultra-large drug library on the Y-axis and percentage of samples for target protein AmpC, on the X-axis. The trained machine learning model is applied on an ultra-large drug library to explore chemical space by identifying one set of top hit molecules by docking against the target protein AmpC. At 902, the graph represents the fraction of the top 500 sampled molecules that are actual top hit molecules, when the top 500 hit molecules are sampled using the whole dataset for target protein AmpC. At 904, the graph represents a fraction of the top 500 sampled molecules that are actual top hit molecules, when top 500 hit molecules are sampled using Mol2Vec for target protein AmpC.

FIG. 9B illustrates a distribution plot of docking scores for the top 1000 hit molecules for target protein AmpC using an ultra-large drug library according to some embodiments herein. The graphical representation depicts probability density on Y-axis and docking score on X-axis. The docking scores for top 1000 hit molecules when top 500 hit molecules are sampled using a random model are shown in the distribution plot of 906. The docking scores for top 1000 hit molecules when top 500 hit molecules are sampled using Mol2Vec are shown in the distribution plot of 908.

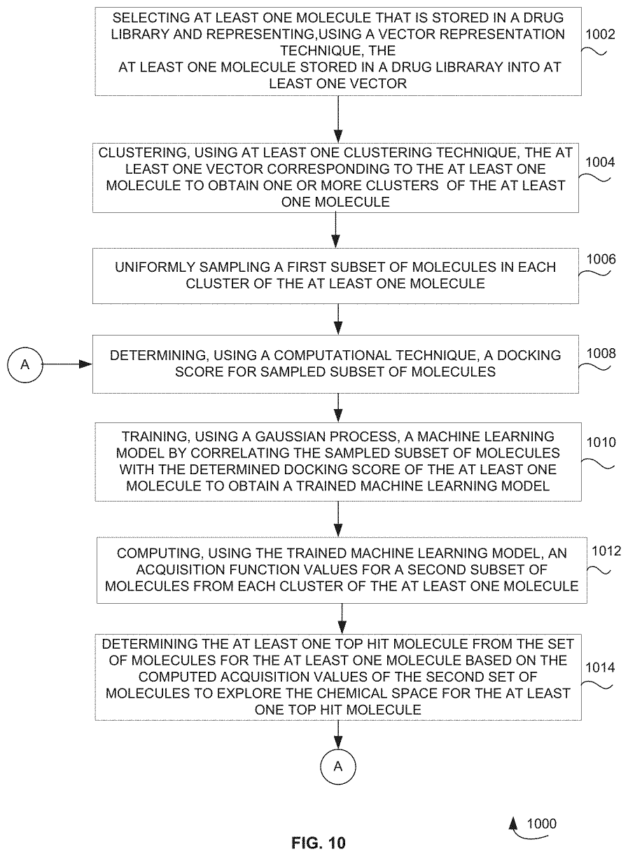

FIG. 10 is a flow diagram that illustrates a method for exploring a chemical space for at least one molecule during molecular design using a machine learning model, according to some embodiments herein. At a step 1002, the method includes selecting the at least one molecule that is stored in a drug library and representing, using a vector representation technique, the at least one molecule as at least one vector. At a step of 1004, the method includes clustering, using at least one clustering technique, the at least one vector corresponding to the at least one molecule to obtain one or clusters of the at least one molecule. At a step of 1006, the method includes uniformly sampling a first subset of molecules in each cluster of the at least one molecule. At a step of 1008, the method includes determining, using a computational technique, a docking score for a sampled subset of molecules. In some embodiments, the docking score determines an acquisition function of the at least one molecule based on the sampled subset of molecules. At a step of 1010, the method includes training, using a gaussian process, the machine learning model by correlating the sampled subset of molecules with the determined docking score of the at least one molecule to obtain a trained machine learning model. At a step 1012, the method computing, using the trained machine learning model, an acquisition function values for a second subset of molecules from each cluster of the at least one molecule. At a step 1014, the method includes determining the at least one top hit molecule for the at least one molecule based on the computed acquisition function values of the second subset of molecules to explore the chemical space for the at least one top hit molecule.

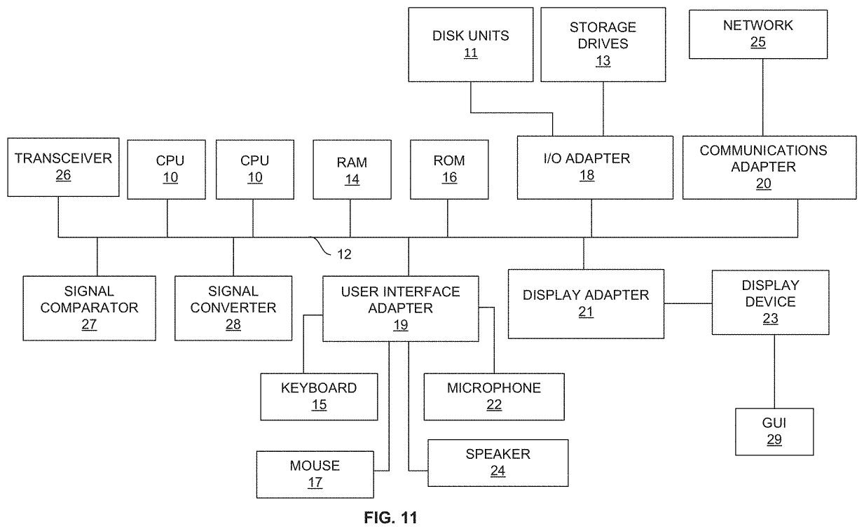

A representative hardware environment for practicing the embodiments herein is depicted in FIG. 11, with reference to FIGS. 1 through 10. This schematic drawing illustrates a hardware configuration of a server 108/computer system/computing device in accordance with the embodiments herein. The system includes at least one processing device CPU 10 that may be interconnected via system bus 14 to various devices such as a random access memory (RAM) 12, read-only memory (ROM) 16, and an input/output (I/O) adapter 18. The I/O adapter 18 can connect to peripheral devices, such as disk units 38 and program storage devices 40 that are readable by the system. The system can read the inventive instructions on the program storage devices 40 and follow these instructions to execute the methodology of the embodiments herein. The system further includes a user interface adapter 22 that connects a keyboard 28, mouse 30, speaker 32, microphone 34, and/or other user interface devices such as a touch screen device (not shown) to the bus 14 to gather user input. Additionally, a communication adapter 20 connects the bus 14 to a data processing network 42, and a display adapter 24 connects the bus 14 to a display device 26, which provides a graphical user interface (GUI) 36 of the output data in accordance with the embodiments herein, or which may be embodied as an output device such as a monitor, printer, or transmitter, for example.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

What is claimed is:

1. A processor-implemented method for exploring a chemical space for at least one molecule during molecular design using a machine learning model, said method comprising:
selecting the at least one molecule that is stored in a drug library and representing, using a vector representation technique, the at least one molecule as at least one vector;
clustering, using at least one clustering technique, the at least one vector corresponding to the at least one molecule to obtain a plurality of clusters of the at least one molecule;
uniformly sampling a first subset of molecules in each cluster of the at least one molecule;
determining, using a computational technique, a docking score for sampled subset of molecules, wherein the docking score determines an acquisition function of the at least one molecule based on the sampled subset of molecules;
training, using a gaussian process, the machine learning model by correlating the sampled subset of molecules with the determined docking score of the at least one molecule to obtain a trained machine learning model;
computing, using the trained machine learning model, the acquisition function values for a second subset of molecules from each cluster of the at least one molecule; and
determining at least one top hit molecule for the at least one molecule based on the computed acquisition function values of the second subset of molecules to explore the chemical space for the at least one top hit molecule.

2. The processor-implemented method of claim 1, wherein representing the at least one molecule into the at least one vector comprises:
extracting a substructure for the at least one molecule at radii 0 and 1 and assigning a unique identifier to the at least one molecule;
representing the at least one molecule as a sentence, using an assigned unique identifier to the at least one molecule; and
encoding words in the sentence for the at least one molecule into the at least one vector using an unsupervised machine learning model, wherein the unsupervised machine learning model is trained by correlating the words for the at least one molecule and the at least one vector.

3. The processor-implemented method of claim 1, further comprises obtaining, using a computational technique, the docking score for the sampled subset of molecules by,
obtaining a structure for the ligand using a dataset, wherein the dataset is obtained from a database, wherein the ligand is an ion or a molecule that binds to a target protein;
obtaining the target protein from a protein database;
performing protein-ligand docking for obtained structure for the ligand and obtained target protein to generate grid maps, electron density, and desolvation maps for each type of atom of each molecule of the sampled subset of molecules; and
computing the docking score for each molecule of the sampled subset of molecules based on generated grid maps, electron density, and desolvation maps for each type of atom.

4. The processor-implemented method of claim 1, wherein computing the acquisition function for the second subset of molecules based on an upper confidence bound, an expected improvement, a probability of improvement obtained from the gaussian process.

5. The processor-implemented method of claim 1, wherein sampling the first subset of molecules uniformly by selecting the at least one top hit molecule based on the value of the acquisition function for the second subset of molecules.

6. The processor-implemented method of claim 1, wherein retraining the machine learning model when convergence criteria are not met, wherein the convergence criteria comprise a maximum number of allowable docking scores for the sampled subset of molecules.

7. One or more non-transitory computer-readable storage medium storing the one or more sequence of instructions, which when executed by the one or more processors, causes to perform a method of enabling a user to explore a chemical space for at least one molecule during molecular design using a machine learning model, wherein the method comprises:
selecting the at least one molecule that is stored in a drug library and representing, using a vector representation technique, the at least one molecule as at least one vector;
clustering, using at least one clustering technique, the at least one vector corresponding to the at least one molecule to obtain a plurality of clusters of the at least one molecule:
uniformly sampling a first subset of molecules in each cluster of the at least one molecule;
determining, using a computational technique, a docking score for sampled subset of molecules, wherein the docking score determines an acquisition function of the at least one molecule based on the sampled subset of molecules;
training, using a gaussian process, the machine learning model by correlating the sampled subset of molecules with the determined docking score of the at least one molecule to obtain a trained machine learning model;
computing, using the trained machine learning model, the acquisition function values for a second subset of molecules from each cluster of the at least one molecule; and
determining at least one top hit molecule for the at least one molecule based on the computed acquisition function values of the second subset of molecules to explore the chemical space for the at least one top hit molecule.

8. A system for exploring a chemical space for at least one molecule during molecular design using a machine learning model, the system comprising:
a device processor; and
a non-transitory computer-readable storage medium storing one or more sequences of instructions, which when executed by the device processor, causes:
selects the at least one molecule that is stored in a drug library and represents, using a vector representation technique, the at least one molecule as at least one vector;
clusters, using at least one clustering technique, the at least one vector corresponding to the at least one molecule to obtain a plurality of clusters of the at least one molecule;
uniformly samples a first subset of molecules in each cluster of the at least one molecule;
determines, using a computational technique, a docking score for sampled subset of molecules, wherein the docking score determines an acquisition function of the at least one molecule based on the sampled subset of molecules;
trains, using a gaussian process, the machine learning model by correlating the sampled subset of molecules with the determined docking score of the at least one molecule to obtain a trained machine learning model;
computes, using the trained machine learning model, the acquisition function values for a second subset of molecules from each cluster of the at least one molecule; and
determines at least one top hit molecule for the at least one molecule based on the computed acquisition function values of the second subset of molecules to explore the chemical space for the at least one top hit molecule.

9. The system of claim 8, wherein representing the at least one molecule into the at least one vector comprises,
extracting a substructure for the at least one molecule at radii 0 and 1 and assigning a unique identifier to the at least one molecule;
representing the at least one molecule as a sentence, using an assigned unique identifier to the at least one molecule; and
encoding words in the sentence for the at least one molecule into the at least one vector using an unsupervised machine learning model, wherein the unsupervised machine learning model is trained by correlating the words for the at least one molecule and the at least one vector.

10. The system of claim 8, further comprises obtaining, using a computational technique, the docking score for the sampled subset of molecules by,
obtaining a structure for the ligand using a dataset, wherein the dataset is obtained from a database, wherein the ligand is an ion or a molecule that binds to a target protein;
obtaining the target protein from a protein database;
performing protein-ligand docking for obtained structure for the ligand and obtained target protein to generate grid maps, electron density, and desolvation maps for each type of atom of each molecule of the sampled subset of molecules; and
computing the docking score for each molecule of the sampled subset of molecules based on generated grid maps, electron density, and desolvation maps for each type of atom.

11. The system of claim 8, wherein computing the acquisition function for the second subset of molecules based on an upper confidence bound, an expected improvement, a probability of improvement obtained from the gaussian process.

12. The system of claim 8, wherein sampling the sampled subset of molecules uniformly by selecting the at least one top hit molecule based on the value of the acquisition function for the second subset of molecules.

13. The system of claim 8, wherein retraining the machine learning model when convergence criteria are not met, wherein the convergence criteria comprise a maximum number of allowable docking scores for the sampled subset of molecules.
